(19) United States
(12) Patent Application Publication
(10) Pub. No.: US 2017/0158836 A1
(43) Pub. Date: Jun. 8, 2017

(54) ORDERED MACROPOROUS HYDROGELS FOR BIORESPONSIVE PROCESSES

(71) Applicant: Carnegie Mellon University, Pittsburgh, PA (US)

(72) Inventors: Krzysztof Matyjaszewski, Pittsburgh, PA (US); Saadyah Averick, Pittsburgh, PA (US); Hongkun He, Pittsburgh, PA (US)

(21) Appl. No.: 15/039,219

(22) PCT Filed: Nov. 25, 2014
(86) PCT No.: PCT/US14/67400

§ 371 Date: May 25, 2016

Related U.S. Application Data
(60) Provisional application No. 61/963,171, filed on Nov. 25, 2013.

(51) Int. Cl. C08J 9/26; C02F 3/34; C12N 9/96; C08J 9/00; C08J 9/36

(52) U.S. Cl. CPC C08J 9/26; C08J 9/0061; C08J 9/36; C12N 9/96; C12Y 304/21004; C02F 3/342; C08J 2201/0462; C08J 2205/022; C08J 2335/02; C08J 2333/12; C08J 2489/00

(57) ABSTRACT

A three-dimensionally ordered macroporous hydrogel for immobilizing a selected bioresponsive molecule and method of making are disclosed. The three-dimensionally ordered macroporous hydrogel comprises a crosslinked polymer that has a system of interconnected pores. The interconnected pores have a uniform pore size in the range of 50 to 5000 nm, and a plurality of first pore functional groups. The plurality of first pore functional groups is selected to immobilize a selected bioresponsive molecule. Examples of bioresponsive molecules include an enzyme; a molecule for: a protein scaffold, solid phase synthesis, nucleic acid synthesis, polypeptide synthesis, analyte detection, adsorption of analytes and measuring analyte concentrations, organic synthesis, and degradation of biologically active agents in wastewater. A method includes forming a colloidal crystal template, polymerizing a hydrogel within the pores of the colloidal crystal template, and selectively removing the colloidal crystal template. The hydrogel can be polymerized using CRP, ATRP and FRP polymerization processes.

### PRIORITY

This application claims the benefit of U.S. Provisional Patent Application Ser. No. 61/963,171 filed Nov. 25, 2013, which is incorporated by reference herein in its entirety.

### GOVERNMENT RIGHTS

This invention was made with government support provided by the National Science Foundation (DMR 09-69301). NMR instrumentation at CMU was partially supported by NSF (CHE-1039870) and the government has certain rights in this invention.

### TECHNICAL FIELD

The present disclosure is directed to novel three-dimensionally ordered macroporous (3DOM) hydrogels with surface functionality designed to immobilize selected enzymes and other bioresponsive molecules, methods of producing the 3DOM hydrogels, and methods of using the same.

### BACKGROUND

Hydrogels are a class of hydrophilic polymers that are crosslinked either physically or chemically to maintain a three-dimensional (3D) structure. (See, Mathur A M, Moorjani S K, and Scranton A B. Journal of Macromolecular Science-Reviews in Macromolecular Chemistry and Physics 1996; C36(2):405-430, which is incorporated herein by reference.) Many synthetic polymers including (polyethylene oxide (PEO), polylactic acid (PLA), polyacrylic acid (PAA), poly(vinyl alcohol), poly(acrylamide), poly(N-vinyl pyrrolidone), poly(hydroxyalkyl (meth)acrylates), (meth)acrylates, and natural biomacromolecules have been utilized to form hydrogels. Due to their biocompatibility and high water absorption capacity, hydrogels have been used in many applications, including wound dressing, drug delivery, controlled-release devices, agriculture, trans-dermal systems, dental materials, implants, ophthalmic applications, injectable polymeric systems, scaffolds for tissue engineering drug delivery, cell carriers and/or entrapment, wound management and tissue engineering, chromatographic packing, electrophoresis gels, pharmaceutical formulations, and tissue sealants. (See, Tissue Engineering Part B—Reviews 2010; 16(4):371-383; Science 2012; 336(6085):1124-1128; Advanced Drug Delivery Reviews 2012; 64:18-23; Polymers 2012; 4(2):997-1011; European Journal of Pharmaceutics and Biopharmaceutics 2000; 50(1):27-46; Tissue Engineering Part A 2009; 15(7):1695-1707; Proceedings of the National Academy of Science of the United States of America 2006; 103(8):2512-2517; Nature Materials 2005; 4(7):518-524; Journal of Applied Polymer Science 2009; 112(4):2261-2269; AAPS PharmSciTech 2007; 8(1):21-21; Biotechnology and Bioengineering 2013; 110(1):318-326, and Journal of Materials Chemistry B 2013; 1(4):485-492, each of which is incorporated herein by reference.)

The ability to control the micro- and macroscopic structure and properties of hydrogels are crucial for optimizing their performance in targeted applications. However, rational designing of hydrogels with desired structure and properties is an exacting task when targeting specific applications. This poses a significant synthetic challenge on how to optimally incorporate the desired features into the hydrogel in order to realize their potential in the desired application. For some applications involving mass transfer or separations such as protein digestion, porous hydrogels are preferred, because porous structure can provide large surface area and good permeability for fluids. The preparation of porous hydrogels with controllable pore size and good pore-pore interconnection are important parameters that are needed to achieve high performance in such special applications listed above. In addition, functionalized hydrogels with specially designed chemical structures are needed to provide them with certain chemical or physical properties.

Colloidal crystals have a structure of three-dimensionally (3D) periodic lattices, which are assembled from monodisperse spherical particles. (See, Adv. Mater. 2000; 12(10):693-713, which is incorporated herein by reference.) Because of their unique structure, colloidal crystals have been used as templates for the preparation of porous materials with highly ordered porous structures. (See, Angew. Chem. Int. Ed. 2011; 50(2):360-388; J Mater. Chem. 2006; 16(7):637-648; Adv. Mater. 2006; 18(16):2073-2094; Chem. Rev. 2012, 112, 3959; Curr. Opin. Solid State Mater. Sci. 2001; 5(6):553-564, each of which is incorporated herein by reference.)

### SUMMARY

In one non-limiting embodiment of the present disclosure, a functionalized three-dimensionally ordered hydrogel that includes a plurality of functional group on the pore surfaces of the hydrogels are disclosed. In one non-limiting embodiment, desired functional groups can be introduced onto the pore surfaces of the hydrogel by the use of functional monomers or comonomers in the formation of the hydrogels or by post polymerization modification of the plurality of first pore functional groups. At least one of the pore functional groups on the pore surface can chemically or physically react with functional groups on a selected bioresponsive molecule to bind or immobilize the bioresponsive molecule to the hydrogel. The functionalized hydrogels comprising a bioresponsive molecule can be used in a number of bio-related applications including, for example, but not limited to, forming a protein scaffold, protein digestion, protein catalysis, solid phase synthesis, nucleic acid synthesis, polypeptide and protein synthesis, organic synthesis, protein purification, analyte detection, adsorption of analytes and measuring analyte concentrations, degradation of biologically active agents in waste water, as noted in the detailed description section.

According to an aspect of the present disclosure, a three-dimensionally ordered macroporous (3DOM) hydrogel comprises a polymer comprising at least one hydrophilic monomer and at least one crosslinker. The polymer comprises a system of interconnected pores. In non-limiting embodiments, the interconnected pores comprise a uniform pore size in the range of 50 to 5000 nm, or in the range of 100 to 1000 nm. The interconnected pores of the hydrogel comprise a plurality of first pore functional groups. At least one of the plurality of first pore functional groups is selected and is accessible to immobilize or covalently bond with a selected bioresponsive molecule. In a non-limiting embodiment, the first pore functional group may be one of a hydroxyl group, a carboxyl group, an amino group, a mercapto group, a nitro group, a cyano group, an azido group, an alkyl group, a halogenoalkyl group, an alkenyl group, an alkenyloxy group, an alkynyl group, an alkoxy group, an alkylthio group, a formyl group, an alkanoyl group, an alkyloxycarbonyl group, an oxo group, an urea group, a thiourea group, an aminoalkyl group, an aryl group, an aralkyl group, an aryloxy group, an arylthio group, an alkylsulfonyl group, an arylsulfonyl group, a carbamoyl, a heterocyclic group, a protected amino, a protected hydroxyl, and a protected carboxyl group.

In a non-limiting embodiment, the hydrophilic monomers of a three-dimensionally ordered macroporous hydrogel according to the present disclosure are selected from the group consisting of (ethylene glycol) (meth)acrylate, hydroxylated-(ethylene glycol) (meth)acrylate, quaternized 2-(dimethylamino)ethyl (meth)acrylate, hydroxyalkyl (meth)acrylates, n-vinyl pyrrolidone, and acrylamides.

In another non-limiting embodiment of the present disclosure, the at least one crosslinker of the three-dimensionally ordered macroporous hydrogel according to the present disclosure comprises a monomeric unit that is selected from the group consisting of (ethylene glycol) di(meth)acrylate, hydroxylated-(ethylene glycol) di(meth)acrylate, quaternized 2-(dimethylamino)ethyl di(meth)acrylate, a hydroxyalkyl di(meth)acrylate, and a diacrylamide.

In another non-limiting embodiment, the at least one crosslinker of a three-dimensionally ordered macroporous hydrogel according to the present disclosure comprises two or more vinyl groups prior to crosslinking with the at least one hydrophilic monomer. In an embodiment, the crosslinker of a three-dimensionally ordered macroporous hydrogel that comprises two or more vinyl groups prior to crosslinking is selected from the group consisting of diethylene glycol di(meth)acrylate, poly(ethyleneoxide) di(meth)acrylate, trimethylolpropane tri(meth)acrylate, a propylene glycol di(meth)acrylate, a diacrylate of hydrophilic polymer, a diacrylate of caprolactone modified hydroxy pivalic acid neopentyl glycol ester, a polyethoxified tetramethylol methane tetraacrylate, a diacrylate, neopentyl glycol di(meth)acrylate, stearyl diacrylate, 1,4-butane diol di(meth)acrylate, and a degradable crosslinker such as bis(2-methacyloyloxyethyl) disulfide. In a specific non-limiting embodiment, a novel three-dimensionally ordered macroporous (3DOM) hydrogel with the immobilized enzyme trypsin was synthesized and used in protein digestion.

According to another aspect of the present disclosure a method of preparing a three-dimensionally ordered macroporous hydrogel is disclosed. In a non-limiting embodiment, a method includes preparing a colloidal crystal template that comprises providing a plurality of spherical particles. The spherical particles have a uniform particle size distribution with an average particle size diameter in the range of 10 nm to 1 μm. The spherical particles are assembled into a colloidal crystal template by one or more processes of sedimentation, centrifugation, electro deposition, vertical deposition, filtration, and slit filling. The colloidal crystal template comprises an ordered and repeating three dimensional array of the spherical particles that define a uniform array of pores with interconnecting pore-pore porosity arising from contacting spherical particles. Each of the pores in the system of interconnected pores has a uniform pore size, wherein the average uniform pore size is in the range of 50 to 5000 nm.

Polymer precursors are infiltrated into the voids of the colloidal crystal template. The polymer precursors comprise at least one hydrophilic monomer and at least one crosslinker. At least one of the polymer precursors comprises a first pore functional group that can form covalent bonds with a selected bioresponsive molecule. The polymer precursors are polymerized within the voids of the colloidal crystal template. After copolymerization and formation of the hydrogel the colloidal crystal template is selectively removed by dissolution so that a three-dimensionally ordered macroporous hydrogel having pore surface functional groups of the present disclosure remains.

### DESCRIPTION OF THE DRAWINGS

The various embodiments of the present disclosure may be better understood when read in conjunction with the following figures in which:

FIG. 1 represents a scheme of the preparation of 3DOM hydrogels by colloidal crystal templating via aqueous ATRP of OEOMA and the SEM images of the resulting materials after drying in vacuum;

FIG. 2 represents a scheme of the preparation of 3DOM hydrogels by colloidal crystal templating via aqueous ATRP of QDMAEMA and the SEM images of the resulting materials after drying in vacuum;

FIG. 3 represents a scheme of the preparation of 3DOM hydrogels by colloidal crystal templating via aqueous FRP of PEODMA, the method used to confirm the presence of pores in the 3DOM hydrogels by FRP of divinylbenzene (DVB) in situ in the pores, and the SEM images of the resulting materials;

FIG. 4 includes photos of 3DOM hydrogel-trypsin in BAPNA solution in a UV cuvette at (a) 0 h and (b) 3 h. (c) the UV-vis spectra from 0 to 3 h.

FIG. 5 is a comparison of the bovine serum albumin (BSA) solution (0.2 mg/mL) before and after being loading with 3DOM hydrogel-trypsin for 1 d. Photos (a,b) and UV-vis spectra (c,d) of 10 μL of the BSA solution in 0.5 mL of coomassie dye-based (Bradford) protein assays (a,c) without loading with 3DOM hydrogel-trypsin and (b,d) loading with 3DOM hydrogel-trypsin for 1 d;

FIG. 6 is 3D XRM image (orthoviews of the reconstructed volume) of the 3DOM hydrogel-trypsin measured in situ with the sample soaked in water;

FIG. 7 is a calibration curve (black line) for the determination of trypsin concentrations in the reaction solution; the red dot represented the sample prepared by diluting the final reaction mixture to 0.25 of its original concentration by adding TRIS buffer solution; and

FIG. 8 is a calibration curve for the determination of trypsin concentrations in the performed leaching experiment.

### DETAILED DESCRIPTION

It is to be understood that certain descriptions of the embodiments described herein have been simplified to illustrate only those elements, features, and aspects that are relevant to a clear understanding of the disclosed embodiments, while eliminating, for purposes of clarity, other elements, features, and aspects. Persons having ordinary skill in the art, upon considering the present description of the disclosed embodiments, will recognize that other elements and/or features may be desirable in a particular implementation or application of the disclosed embodiments. However, because such other elements and/or features may be readily ascertained and implemented by persons having ordinary skill in the art upon considering the present description of the disclosed embodiments, and are therefore not necessary for a complete understanding of the disclosed embodiments, a description of such elements and/or features is not provided herein. As such, it is to be understood that the description set forth herein is merely exemplary and illustrative of the disclosed embodiments and is not intended to limit the scope of the invention as defined solely by the claims.

Any numerical range recited herein is intended to include all sub-ranges subsumed therein. For example, a range of “1 to 10” or “from 1 to 10” is intended to include all sub-ranges between (and including) the recited minimum value of 1 and the recited maximum value of 10, that is, having a minimum value equal to or greater than 1 and a maximum value of equal to or less than 10. Any maximum numerical limitation recited herein is intended to include all lower numerical limitations subsumed therein and any minimum numerical limitation recited herein is intended to include all higher numerical limitations subsumed therein. Accordingly, applicants reserve the right to amend the present disclosure, including the claims, to expressly recite any sub-range subsumed within the ranges expressly recited herein. All such ranges are intended to be inherently disclosed herein such that amending to expressly recite any such sub-ranges would comply with the requirements of 35 U.S.C. §112(a), and 35 U.S.C. §132(a).

The grammatical articles “one”, “a”, “an”, and “the”, as used herein, are intended to include “at least one” or “one or more”, unless otherwise indicated. Thus, the articles are used herein to refer to one or more than one (i.e., to at least one) of the grammatical objects of the article. By way of example, “a component” means one or more components, and thus, possibly, more than one component is contemplated and may be employed or used in an implementation of the described embodiments.

All percentages and ratios are calculated based on the total weight of the particular material composition, unless otherwise indicated.

Any patent, publication, or other disclosure material that is said to be incorporated, in whole or in part, by reference herein is incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference. Any material, or portion thereof, that is said to be incorporated by reference herein, but which conflicts with existing definitions, statements, or other disclosure material set forth herein is only incorporated to the extent that no conflict arises between that incorporated material and the existing disclosure material.

The present disclosure includes descriptions of various embodiments. It is to be understood that all embodiments described herein are exemplary, illustrative, and non-limiting. Thus, the invention is not limited by the description of the various exemplary, illustrative, and non-limiting embodiments. Rather, the invention is defined solely by the claims, which may be amended to recite any features expressly or inherently described in or otherwise expressly or inherently supported by the present disclosure.

According to an aspect of the present disclosure, a three-dimensionally ordered macroporous hydrogel having a system of interconnected pores with reactive pore functional groups is disclosed. In a non-limiting embodiment, a three-dimensionally ordered macroporous hydrogel is comprised of a polymer that contains at least one hydrophilic monomer and at least one crosslinker that crosslinks the at least one hydrophilic monomer, rendering the crosslinked hydrogel resistant to dissolution by organic solvents and certain inorganic acids.

In a non-limiting embodiment, the polymer of the hydrogel comprises a system of interconnected pores, where each pore of the system of interconnected pores has a uniform pore size in the range of 50 to 5000 nm. In another non-limiting embodiment the uniform pore size is in the range of 100 to 1000 nm. Hydrogels having pores with uniform pore sizes in the disclosed ranges are referred to herein as “macroporous” hydrogels.

The interconnected pores of the three-dimensionally ordered macroporous hydrogels according to the present disclosure comprise a plurality of first pore functional groups on the pore surfaces. In a non-limiting embodiment, the first pore functional group is selected to covalently bond with a selected bioresponsive molecule, such as, but not limited to an enzyme. In a non-limiting embodiment at least one of the plurality of first pore functional groups is bonded to the selected bioresponsive molecule.

As used herein, the term “three-dimensionally ordered” indicates that the interconnected pores of the macroporous hydrogel disclosed herein are arranged in a regular array that repeats in three-dimensions.

In a non-limiting embodiment, the at least one hydrophilic monomer of the three-dimensionally ordered macroporous hydrogel of the present disclosure is selected from the group consisting of (ethylene glycol) (meth)acrylate, hydroxylated-(ethylene glycol) (meth)acrylate, quaternized 2-(dimethylamino)ethyl (meth)acrylate, hydroxyalkyl (meth)acrylates, n-vinyl pyrrolidone, and acrylamides. It will be recognized that the term “monomer” as used herein, also refers to and includes oligomers and polymers of the recited monomer. It will also be recognized that the prefix “(meth)” for a chemical species indicates that that chemical species can be a methylated or non-methylated species. For example the term “(meth)acrylate” includes methacrylate and the acrylate versions of that chemical species or compound.

The three-dimensionally ordered macroporous hydrogel disclosed herein, in a non-limiting embodiment, includes at least one crosslinker that comprises a monomeric unit that is selected from the group consisting of (ethylene glycol) di(meth)acrylate, hydroxylated-(ethylene glycol) di(meth)acrylate, quaternized 2-(dimethylamino)ethyl di(meth)acrylate, a hydroxyalkyl di(meth)acrylate, and a diacrylamide. The crosslinkers may be in a monomeric, oligomeric, or polymeric form.

In another non-limiting embodiment, the three-dimensionally ordered macroporous hydrogel comprises at least one crosslinker that contains two or more vinyl groups, prior to crosslinking. Such crosslinker may be selected from, but are not limited to the group consisting of diethylene glycol di(meth)acrylate, poly(ethyleneoxide) di(meth)acrylate, trimethylolpropane tri(meth)acrylate, a propylene glycol di(meth)acrylate, a diacrylate of hydrophilic polymer, a diacrylate of caprolactone modified hydroxy pivalic acid neopentyl glycol ester, a polyethoxified tetramethylol methane tetraacrylate, a diacrylate, neopentyl glycol di(meth)acrylate, stearyl diacrylate, 1,4-butane diol di(meth)acrylate, and bis(2-methacyloyloxyethyl) disulfide. In a specific embodiment the at least one crosslinker comprises a substituted divinylbenzene. Not meant to be limiting, the crosslink density in the matrix of the hydrogel according to the present disclosure may be between 1-100% of the vinyl units present in hydrogel.

The pore surfaces of the interconnected pores of the three-dimensionally ordered macroporous hydrogel of the present disclosure contain a plurality of chemical functional groups, or simply, “functional groups”. The functional groups are selected to react with a bioresponsive molecule in order to facilitate various biological functions, such as, for example, protein digestion. The functional groups may be initially present on the at least one hydrophilic monomer or the crosslinker, and these are referred to herein as a “first pore functional group”. Alternatively, the first pore functional groups may be chemically reacted with a one or more different chemical species to change the first pore functional groups into one or more differing functional groups. Pore functional groups that are derived from the first pore functional groups are referred to herein as “second pore functional groups”. Methods of reacting a chemical species to convert a first pore functional group into a second pore functional group are known to a person having ordinary skill in the art and need not be elaborated upon herein.

In a non-limiting embodiment of the three-dimensionally ordered macroporous hydrogel disclosed herein the first pore functional groups or the second pore functional groups are selected from the group consisting of a hydroxyl group, a carboxyl group, an amino group, a mercapto group, a nitro group, a cyano group, an azido group, an alkyl group, a halogenoalkyl group, an alkenyl group, an alkenyloxy group, an alkynyl group, an alkoxy group, an alkylthio group, a formyl group, an alkanoyl group, an alkyloxycarbonyl group, an oxo group, an urea group, a thiourea group, an aminoalkyl group, an aryl group, an aralkyl group, an aryloxy group, an arylthio group, an alkylsulfonyl group, an arylsulfonyl group, a carbamoyl, a heterocyclic group, a protected amino, a protected hydroxyl, and a protected carboxyl group.

In a non-limiting embodiment of the three-dimensionally ordered macroporous hydrogel of the present disclosure, at least one of the plurality of first or second pore functional groups can be utilized to form a covalent bond with one or more selected bioresponsive molecules. The number of the plurality of functional groups on the pore surfaces that can be utilized to form covalent bonds with a bioresponsive molecule is only limited by the size of the selected bioresponsive molecule in relation to the uniform pore size, and the resultant accessibility of the bioresponsive molecule to the functional groups on the pore surfaces.

In a non-limiting embodiment of the three-dimensionally ordered macroporous hydrogel of the present disclosure, a fraction in the range of 1-100% of first pore functional groups can be converted to a plurality of one or more differing second pore functional groups and the formed mixture of functional groups are utilized to form covalent bonds with one or more selected bioresponsive molecules.

In a non-limiting embodiment, at least one of the first or at least one of the second pore functional groups is covalently bonded with a bioresponsive molecule comprising an enzyme. In a specific embodiment, the bioresponsive molecule comprises the enzyme trypsin.

Bioresponsive molecules that may bond to the pore functional groups of the three-dimensionally ordered macroporous hydrogel according to the present disclosure include, but are not limited to: a bioresponsive molecule for formation of a protein scaffold, a bioresponsive molecule for protein purification; a bioresponsive molecule for solid phase synthesis, a bioresponsive molecule for nucleic acid synthesis, a bioresponsive molecule for polypeptide synthesis, a bioresponsive molecule for analyte detection; a bioresponsive molecule for adsorption of analytes and measuring analyte concentrations, a bioresponsive molecule for organic synthesis, and a bioresponsive molecule for degradation of biologically active agents in wastewater. Examples of the aforementioned bioresponsive molecules and their reactions schemes with specific pore functional groups are known to a person having ordinary skill in the art or could be easily determined without undue experimentation. Therefore, specific details of the aforementioned bioresponsive molecules and their reactions schemes with specific pore functional groups need not be elaborated upon herein.

The hydrogels of the present disclosure, once bound by bioresponsive molecules, may be used for protein purification, solid phase synthesis, nucleic acid synthesis, polypeptide synthesis, analyte detection, adsorption of analytes, measuring analyte concentrations, organic synthesis, and degradation of biologically active agents in wastewater. (See, Tissue Engineering Part B—Reviews 2010; 16(4):371-383; Science 2012; 336(6085):1124-1128; Advanced Drug Delivery Reviews 2012; 64:18-23; Polymers 2012; 4(2):997-1011; European Journal of Pharmaceutics and Biopharmaceutics 2000; 50(1):27-46; Tissue Engineering Part A 2009; 15(7):1695-1707; Proceedings of the National Academy of Science of the United States of America 2006; 103(8):2512-2517; Nature Materials 2005; 4(7):518-524; Journal of Applied Polymer Science 2009; 112(4):2261-2269; AAPS PharmSciTech 2007; 8(1):21-21; Biotechnology and Bioengineering 2013; 110(1):318-326, and Journal of Materials Chemistry B 2013; 1(4):485-492, each of which is incorporated herein by reference.)

In a non-limiting embodiment of the three-dimensionally ordered macroporous hydrogel of the present disclosure, the bioresponsive molecule comprises trypsin, papain protein G or synthetically relevant agents exemplified by Lipase.

Four non-limiting examples are provided for potential proteins that can be immobilized onto the three-dimensionally ordered macroporous hydrogels 1) Trypsin-a serine protease that is used to degrade proteins for sequencing using mass-spectroscopy. 2) Papain-a cysteine protease that is used to digest antibodies to their fragment antigen binding (Fab) and fragment conserved (Fc) units. 3) Protein-G is used to isolate Immunoglobulin G (IgG) by binding to their Fc regions. 4) Lipase-B can selectively catalyze the asymmetric hydrolysis of esters and is therefore applied for the synthesis of optically pure pharmaceuticals.

According to another aspect of the present disclosure, a method of preparing a three-dimensionally ordered macroporous hydrogel is disclosed. The method comprises preparing a colloidal crystal template. A non-liming embodiment to prepare a colloidal crystal template includes providing a plurality of spherical particles. The colloidal particles are uniform in size. That is, it a non limiting embodiment, they have a monodisperse particle distribution, uniformly sized particle having an average particle size diameter in the range of 10 nm to 1 μm; or in other non-limiting embodiments, the spherical particles have a monodisperse particle distribution, having an average particle size diameter in the range of 10 nm to 100 μm, or in a range of 100 to 1000 nm. The spherical particles are assembled into a colloidal crystal template. Assembling processes include a process of one or more of sedimentation, centrifugation, electro deposition, vertical deposition, filtration, and slit filling. The resulting colloidal crystal template comprises an ordered and repeating array of the spherical particles that define a uniform array of pores having a uniform pore size between contacting spherical particles. In non-limiting embodiments the average uniform pore size in the uniform array of pores is in the range of 50 to 5000 nm, or in the range of 100 to 1000 nm.

In a non-limiting embodiment, the plurality of polymeric spherical particles comprises silica particles. The preparation of spherical silica particles having a monodisperse particle size distribution in the size ranges disclosed herein is known to a person having ordinary skill in the art and method of preparing such silica particles would not require undue experimentation. At least for this reason, techniques used to prepare monodisperse silica particles that are amenable to the colloidal crystals of the present disclosure need not be further elaborated upon herein.

In another non-limiting embodiment, the plurality of polymeric spherical particles comprises polymeric particles. As described later herein, polymeric spherical particles can be prepared by surfactant free emulsion polymerization, which provides spherical particles having a uniform particle size distribution in the ranges desired for the colloidal crystal templates described herein. In a non-limiting embodiment, the polymeric spherical particles comprise latex particles. In another non-limiting embodiment, the polymeric spherical particles comprise one or more monomeric units selected from the group consisting of, but are not limited to, styrene, methyl (meth)acrylate, tert-butyl(meth)acrylate, n-butyl(meth)acrylate, vinyl acetate, and acrylamide. It will be recognized that a person having ordinary skill in the art understands that these monomers alone, or in combinations, can be polymerized using surfactant free emulsion polymerization techniques to form spherical particles having a uniform particle size distribution in the ranges desired for the colloidal crystal templates described herein. In another non-limiting embodiments, the plurality of polymeric spherical particles comprises one of polystyrene (PS) particles and poly(methyl (meth)acrylate) (PM(M)A) particles. In another non-limiting embodiment the plurality of polymeric spherical particles comprises poly(methylmethacrylate) (PMMA) particles.

A non-limiting method embodiment for assembling the polymeric spherical particles into a colloidal crystal template step comprises centrifuging the polymeric spherical particles.

Hydrophilic (co)polymer precursors are infiltrated into the voids of the colloidal crystal template. In a non-limiting embodiment, polymer precursors comprise at least one hydrophilic monomer and at least one crosslinker. One or more of the (co)polymer precursors comprise a first functional group that can form covalent bonds with a selected bioresponsive molecule. The polymer precursors are polymerized within the void forming network of the colloidal crystal template. After formation of the crosslinked hydrogel the colloidal crystal template is then selectively removed/dissolved, resulting in a three-dimensionally ordered macroporous hydrogel of the present disclosure.

In a non-limiting embodiment of a method disclosed herein, the at least one hydrophilic monomer is selected from the group consisting of (ethylene glycol) (meth)acrylate, hydroxylated-(ethylene glycol) (meth)acrylate, quaternized 2-(dimethylamino)ethyl (meth)acrylate, hydroxyalkyl (meth)acrylates, n-vinyl pyrrolidone, and acrylamides. It will be recognized that oligomeric and polymeric forms of these hydrophilic monomers may be used in the method disclosed herein.

In a non-limiting embodiment of a method disclosed herein, the at least one crosslinker is selected from the group consisting of (ethylene glycol) di(meth)acrylate, hydroxylated-(ethylene glycol) di(meth)acrylate, quaternized 2-(dimethylamino)ethyl di(meth)acrylate, a hydroxyalkyl di(meth)acrylate, and a diacrylamide. It will be recognized that monomeric, oligomeric and polymeric forms of crosslinkers are included in the methods disclosed herein and when, oligomeric and polymeric forms of crosslinkers are utilized the can additionally comprise first functional groups.

In an additional non-limiting embodiment of methods disclosed herein, the at least one crosslinker is selected from the group consisting of diethylene glycol di(meth)acrylate, poly(ethyleneoxide) di(meth)acrylate, trimethylolpropane tri(meth)acrylate, a propylene glycol di(meth)acrylate, a diacrylate of hydrophilic polymer, a diacrylate of caprolactone modified hydroxy pivalic acid neopentyl glycol ester, a polyethoxified tetramethylol methane tetraacrylate, a diacrylate, neopentyl glycol di(meth)acrylate, stearyl diacrylate, 1,4-butane diol di(meth)acrylate, and bis(2-methacyloyloxyethyl) disulfide.

The methods disclosed herein include, but are not limited to, infiltrating polymer precursors into the colloidal crystal template with polymeric precursors required for a controlled radical polymerization (CRP). In a non-limiting embodiment, a method disclosed herein includes infiltrating the colloidal crystal template with polymeric precursors required for an atom transfer radical polymerization reaction (ATRP). In a non-limiting embodiment, the polymeric precursors for ATRP comprise at least one hydrophilic monomer, at least one crosslinker, an initiator, a transition metal catalyst having two accessible oxidation states that are separated by one electron, and a ligand capable of forming a soluble ligand-transition metal catalyst complex. In a specific method, infiltrating the colloidal crystal template comprises infiltrating with an aqueous solution comprising a isobutryl (iB) brominated poly(ethylene glycol) initiator (PEG), oligo(ethylene glycol) methyl ether (meth)acrylate (OEOMA) monomer, poly(ethylene oxide) di(meth)acrylate (PEOMA) crosslinker, cuprous chloride (CuCl), cupric chloride (CuCl₂), and 2,2′-bipyridine (bpy). In still another specific, but not limiting method, the molar ratios of PEG/OEOMA/PEOMA/CuCl/CuCl₂/bpy infiltrated into the pores of the colloidal crystal range from 1/120/8/1/9/21 to 1/120/45/1/9/21, and wherein the monomer to initiator ratio is in a range of 10-10,000 to 1. In another non-limiting embodiment, the step of infiltrating polymer precursors into the colloidal crystal template comprises infiltrating the colloidal crystal template with an aqueous solution comprising a brominated poly(ethylene glycol) initiator (PEG), oligo(ethylene glycol) methyl ether (meth)acrylate (OEOMA) monomer, poly(ethylene oxide) di(meth)acrylate (PEOMA) crosslinker, cuprous halide (CuX), cupric chloride (CuX₂), and a ligand (L) forming a soluble complex with the transition metal catalyst, and the molar ratios of PEG/OEOMA/PEOMA/CuX/CuX₂/L range from 1/120/8/1/9/21 to 1/120/45/1/9/21, and wherein the monomer to initiator ratio is in a range of 10-10,000 to 1.

A method disclosed herein includes infiltrating polymer precursors into the colloidal crystal template with polymeric precursors required for a free radical polymerization reaction. In a specific non-limiting embodiment for free radical polymerization, the polymeric precursors include at least one monomer comprising poly(ethylene glycol (meth)acrylate (PEOMA) and at least one crosslinker comprising poly(ethylene oxide) di(meth)acrylate (PEODMA) and a free radical initiator. Any method disclosed herein may further comprise a comonomer selected from the group consisting of a substituted styrene, a (meth)acrylate, an acrylamide, and a vinyl pyrrolidone.

It will be recognized that the methods disclosed herein may further comprise covalently bonding a plurality of bioresponsive molecules to at least one of the first pore functional groups or at least one of the second pore functional groups of the three-dimensionally ordered macroporous hydrogel. In a specific embodiment, the bioresponsive molecule of a method comprises bonding trypsin to a pore functional group.

The methods of the present disclosure comprise selectively removing the colloidal crystal template from the hydrogel by dissolving the colloidal crystal template in a solvent, where the solvent is selected not to solubilize the three-dimensionally ordered macroporous hydrogel. In a non-limiting embodiment when the colloidal crystal template comprises silica particles, the solvent, or particle removing agent, comprises hydrofluoric acid. In a non-limiting embodiment when the colloidal crystal template comprises polymeric particles, the solvent comprises one or more of acetone, tetrahydrofuran, and a solution of acetone and tetrahydrofuran.

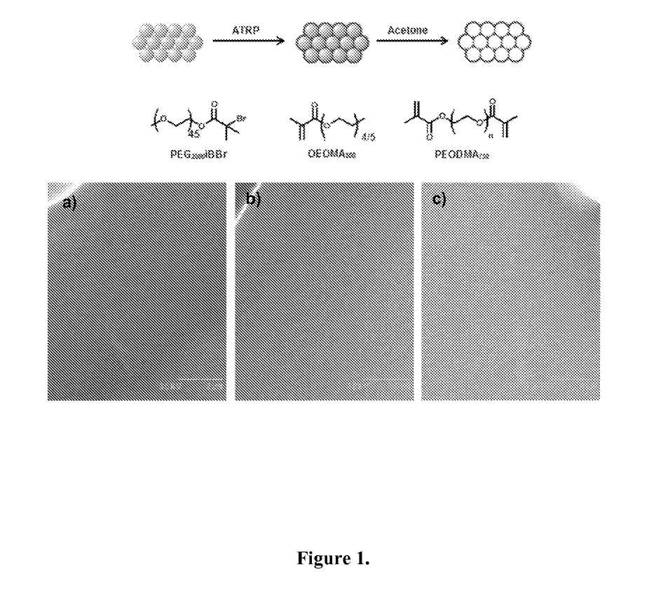

A series of experiments were conducted targeting the preparation of three-dimensionally ordered macroporous (3DOM) hydrogels by aqueous ATRP copolymerization, initially exemplified by the copolymerization of poly(ethylene glycol) (meth)acrylate (PEOMA) and poly(ethylene oxide) di(meth)acrylate (PEODMA) in the presence of a PMMA latex particle based colloidal crystal as the template. In a non-limiting embodiment the PMMA latex particles for formation of the colloidal crystal were synthesized by surfactant-free emulsion polymerization. For the polymerization of the PMMA particles, 165 mL of water, 30 mL of methyl (meth)acrylate (MMA), and 76 mg of 2,2′-azobis(2-methylpropionamidine) dihydrochloride (AAPH) as an azo initiator were combined and stirred. Stirring took place at 350 rpm in a nitrogen atmosphere at 75° C. for 2 h. The PMMA colloidal crystal templates were formed by centrifuging the colloid at 1500 rpm for 24 h, decanting the water, and allowing the solid to dry over 3 days. These PMMA colloidal crystal templates generated a uniform interconnected pore size in a hydrogel of the present disclosure in the range of 100-1000 nm. The PMMA colloidal crystal was removed by dissolution in acetone to generate the 3D ordered macropores with interconnected windows in the crosslinked hydrogel. However, after drying SEM images of the surfaces of the dried hydrogel did not show the presence of pores which was attributed to the collapse of the pores during drying, FIG. 1, images a), b) and c) for hydrogels with 7, 21 and 38% crosslinker respectively.

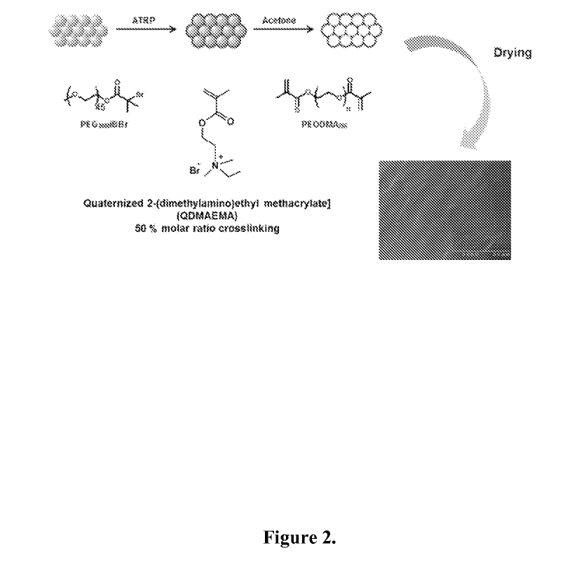

Another drying method was examined, freeze-drying, but there were still no observable pores in the resulting material. Therefore a more rigid water-soluble (meth)acrylate monomer, quaternized 2-(dimethylamino)ethyl (meth)acrylate] (QDMAEMA), was incorporated into the walls of the hydrogel stabilized with 50% molar ratio of the crosslinking agent. However, after drying in vacuum, there were still no pores observed in the resulting solid materials, FIG. 2.

In order to evaluate whether the collapse of the pores may be due to an insufficient degree of crosslinking, a series of control experiments were conducted by a standard free radial polymerization (FRP) with different degrees of crosslinking. Forming the hydrogel by FRP would be expected to generate a less well defined crosslink structure than ATRP, one comprising areas of more densely crosslinked networks and hence a more rigid structure. Nevertheless, there were still no pores observed in the resulting materials after drying in vacuum. The results showed that preserving observable pores after drying was very difficult, so the question appeared to be whether it is possible to isolate the 3DOM without drying.

A critical observation was that after removing the colloidal crystal templates by acetone and before drying, the materials showed green-blue colors when they were soaked in matrix expanding solvents, e.g., acetone, or water. This phenomenon indicated that materials contained ordered 3D arrays of macropores with diameters that were similar to wavelength of light, and that the interconnected pores reformed in the presence of suitable solvents.

In one embodiment of the invention it was determined that the pores in the first formed colloidal crystal templated macroporous hydrogel can be preserved without drying, by using solvent exchange approach as an alternative strategy for isolation of the hydrogel. The resulting porous materials can be used in other conditions for many different applications.

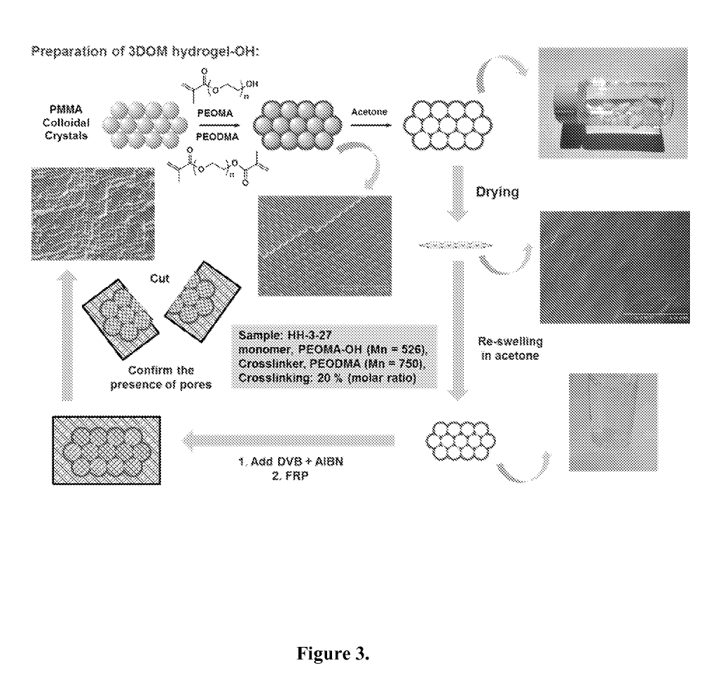

The method that was used to confirm the presence of pores when the material was to disperse the hydrogel in solvent with addition of DVB to the solvent that contained the porous polymers before drying, and then conducting FRP to generate a crosslinked polymer network in situ in the pores of the porous hydrogel. The presence of pores was confirmed by cutting the final crosslinked composite and examining the surface of the cut section of porous polymers by SEM. The SEM images, FIG. 3, showed the presence of ordered packed uniform monodisperse spheres, which were the crosslinked DVB replica of the initial interconnected macropores formed in the colloidal templated porous copolymers thereby confirming the presence of interconnected pores in the first formed hydrogel.

In order to check if the ordered 3D macroporous structure of the hydrogel was retained during multiple drying/re-swelling cycles. The SEM images of the porous polymer after 10 drying/re-swelling cycles was obtained by conducting a FRP of styrene in the presence of differing concentrations of DVB in situ in the pores. The results clearly showed that uniform pores continued to exist in the materials, confirming the good reversibility of the shrinkage and expansion throughout many series of drying and swelling events, FIG. 3.

In order to introduce an exemplary bio-active molecule or agent, also referred to herein as a bioresponsive molecule, into hydrogels of the present disclosure, another monomer, PEOMA-OH (Mₙ=526), was used in the synthesis of the porous hydrogel, and trypsin was introduced onto the surface of the pores of the hydrogel. The hydrogel containing —OH groups can be synthesized by either FRP or aqueous ATRP. Images of the hydrogel containing —OH groups prepared by FRP with a degree of crosslinking of 20% are presented in FIG. 3 where the images are identified as (a) after polymerization, (b) after washing with acetone and drying, and (c) after FRP of DVB in situ in the pores. It is seen that the collapsed pores after drying are reformed in the presence of a solvent allowing DVB crosslinking within the well-defined interlinked network of pores.

In a non-limiting embodiment, exemplary 3DOM hydrogels were prepared by the copolymerization of poly(ethylene glycol) (meth)acrylate (PEOMA) and poly(ethylene oxide) di(meth)acrylate (PEODMA) in the presence of a poly(methyl (meth)acrylate) (PMMA) latex colloidal crystal as the template. Other derivatives of biocompatible hydrophilic polymers, in addition to polyethylene oxide (PEO), such as polylactic acid (PLA), polyacrylic acid (PAA), poly(vinyl alcohol), poly(acrylamide), poly(N-vinyl pyrrolidone), poly(hydroxyalkyl (meth)acrylates) (meth)acrylate, and natural biomacromolecules can be incorporated into monomers of divinyl monomers for use in preparation of the 3DOM hydrogels.

The PMMA latex particles that were assembled into the colloidal crystal were synthesized by surfactant-free emulsion polymerization, and provided a uniform interconnected pore size in the formed hydrogel, typically in the range of 50-5000 nm, after being used as the sacrificial template. After formation of the hydrogel the particles forming the colloidal crystal were dissolved in acetone to generate the 3D ordered structure with the macropores interconnected with windows in the crosslinked hydrogel, which facilitate the transport of liquids through the pores. The porous structure of the hydrogel was able to undergo reversible shrinkage and expansion by drying and swelling in aqueous media.

A non-limiting embodiment included introducing trypsin onto the pore surfaces through condensation reactions with surface accessible complementary functionality. The structure of the functionalized 3DOM hydrogel was characterized by scanning electron microscopy (SEM) and nanoscale 3D X-ray microscopy (XRM).

The hydrogel containing —OH groups was reacted with succinic anhydride to produce surface tethered —COOH group. In the present disclosure, this is a non-limiting example of when a first pore functional group is converted to a second pore functional group that is utilized to form covalent bonds with a selected bio-active agent. The surface tethered —COOH group was then reacted with trypsin in the presence of EDC and N-hydroxysuccinimide (NHS) to immobilize trypsin on the pore surfaces as shown in Scheme 1 producing an exemplary trypsin serine protease simply by conducting small molecule condensation reactions within the pores of the hydrogel.

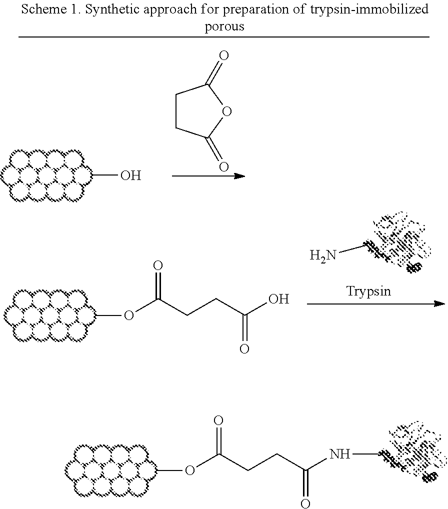

hydrogel.

PEOMA-OH (Mn=750), PEODMA (Mn=750), 20 mol % crosslinking.

The resulting hydrogel-trypsin was tested for enzyme digestion using an aqueous solution of N_{α}-benzoyl-L-arginine p-nitroanilide (BAPNA) or bovine serum albumin (BSA), Scheme 2.

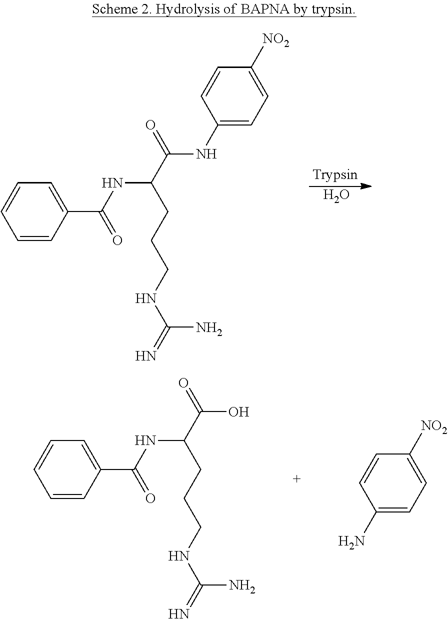

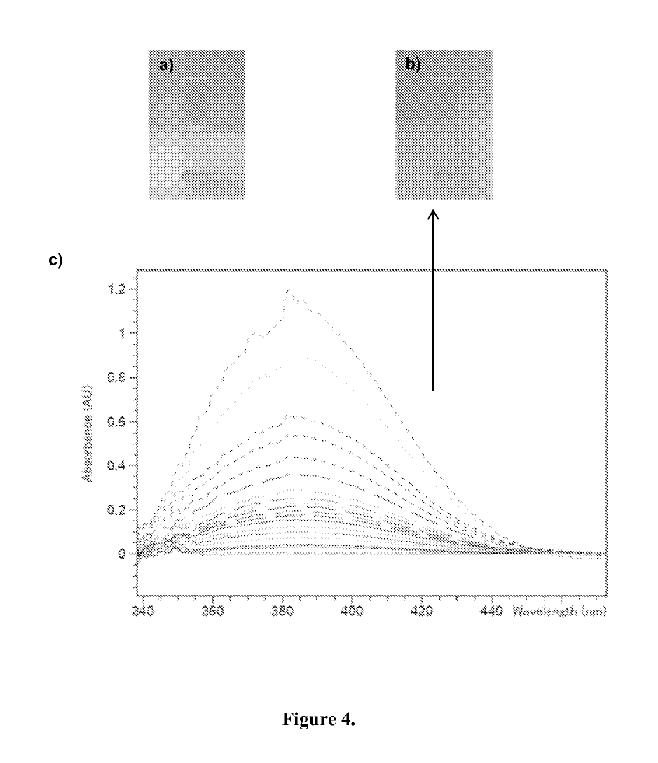

The activity of the immobilized trypsin was determined by observation of the reaction of trypsin with N_{α}-benzoyl-L-arginine p-nitroanilide (BAPNA) in tris buffer (50 mM, pH 8) in a UV quartz cuvette. The 3DOM hydrogel-trypsin was added to the mixture and UV spectra were measured periodically. The gradual increase of the UV absorption peak at 385 nm indicated the gradual increase of the concentration of p-nitroaniline, which is a product of the hydrolysis of BAPNA by trypsin, FIG. 4.

A column was prepared by filling a syringe with 3DOM hydrogel-trypsin. The BAPNA solution was passed through the column and the color of the solution changed from colorless to yellow immediately, indicating that the hydrolysis of BAPNA by hydrogel-trypsin occurred in the column as the solution passed through the hydrogel. The fact that the hydrolysis was successful as shown in FIG. 4 where the cumulative UV-vis spectra recorded over a period of 3 hours is shown.

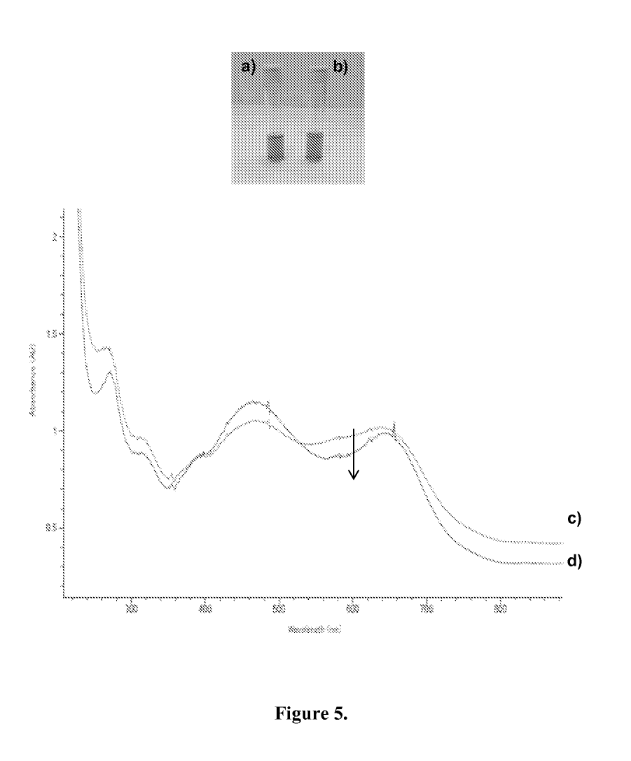

FIG. 5 shows the results of an experiment when the hydrogel-trypsin was mixed with a solution of bovine serum albumin (BSA) for 1 d, and the solution was taken and mixed with coomassie dye-based (Bradford) protein assays and compared to a control sample of pure BSA solution of the same concentration mixed with coomassie dye-based (Bradford) protein assays. The colors of the resulting two solutions are different, the stronger the blue color, the higher the concentration of protein in the solution. UV-vis spectra also showed that the absorption at 600 nm decreased compared to the control sample, indicating some of the BSA has been digested in the presence of the hydrogel-trypsin.

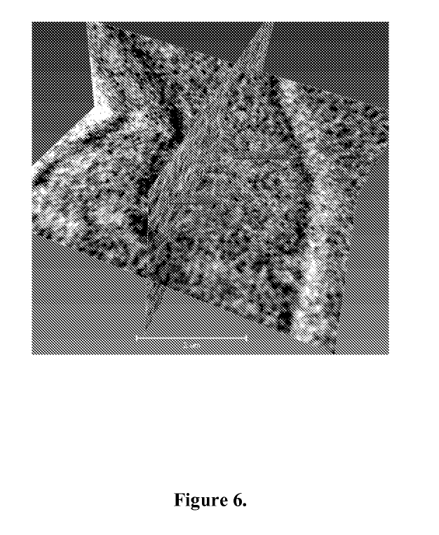

FIG. 6 shows the 3D XRM image (orthoviews of the reconstructed volume) of the 3DOM hydrogel-trypsin measured in situ with the sample soaked in water. The ordered porous structure was visualized and has an average pore size of 0.1 μm. The pore size can be changed by using colloidal crystal templates with different particle sizes.

Other biotechnologically relevant agents that can similarly be tethered to the surface of the hydrogel are exemplified by Papain and protein G and synthetically relevant agents are exemplified by Lipase.

These results show the broad applicability of the disclosed procedure. Crosslinked water swellable 3DOMs can be prepared from a broad spectrum of functional, or functionalizable free radically copolymerizable monomers with a range of crosslink densities employing colloidal crystal templates to provide a system of interconnected pores of the desired dimensions, 50-5000 nm in size, preferentially between 100 and 1000 nm. The accessible functionality can be directly, or after conversion of the first incorporated functionality to a desired functionality, utilized to incorporate a protein or a small molecule for antibody recognition onto the surface of the porous hydrogel.

Since the % of crosslinker that can be incorporated into the hydrogel can be varied over a broad range, from 1-100%, the crosslinker can be selected to incorporate additional functionality to interact with added reactants/agents in addition to being hydrophilic. Furthermore in addition to the crosslinker comprising two or more vinyl units it can comprise one or more segments of non-radically copolymerizable hydrophilic polymers such as PEO and PLA.

Due to their unique structures, colloidal crystals have been used as templates for highly ordered rigid porous polymeric structures in recent years. (See, Chem. Rev. 2012, 112, 3959, which is incorporated by reference herein.) The inverse polymer opals are formed in the interstitial sites of the colloidal crystal templates, giving a three-dimensionally ordered macroporous (3DOM) structure. (See, Colloid Surf. A—Physicochem. Eng. Asp. 2002, 202, 281, which is incorporated by reference herein.).

The preparation of periodic macroporous structures by colloidal crystal templating involves four steps: synthesis of colloidal spheres, preparation of the colloidal crystal template, precursor infiltration followed by polymerization, and template removal. (See, Chem. Mater. 2008, 20, 649, which is incorporated by reference herein.)

Various inorganic or polymeric particles can be used in the first step as long as they can be removed by reactive etching or dissolution in a solvent. Examples include monodisperse silica particles (SiO₂) and polymeric particles, such as, for example, polystyrene (PS) or poly(alkyl)acrylates, include the exemplary poly(methylmethacrylate) (PMMA), or other polymers synthesized from modified styrenic/acrylate/(meth)acrylate monomers, or a mixture of such monomers. (See, Adv. Mater. 2000, 12, 531, which is incorporated by reference herein.)

The second step involves the assembly of the spheres into a colloidal crystal, which can be achieved by various approaches, such as sedimentation (see, Science 1989, 245, 507; Langmuir 1999, 15, 4701; and Soft Matter 2005, 1, 265; each of which is incorporated herein by reference), centrifugation (see, Soft Matter 2005, 1, 265); electro deposition (see, Phys. Rev. Lett. 1989, 63, 2753; which is incorporated herein by reference), vertical deposition (see, Chem. Mater. 1999, 11, 2132; Physica status solidi. A 2007, 204, 3618; and Langmuir 1996, 12, 1303; each of which is incorporated herein by reference), filtration (see, J. Mater. Chem. 2002, 12, 3261; and Chem. Mater. 2002, 14, 3305; each of which is incorporated herein by reference), and slit filling (see, Adv. Mater. 1998, 10, and 1028; Langmuir 1999, 15, 266; each of which is incorporated herein by reference). In a non-limiting embodiment of the present disclosure, a centrifugation measure was used in which PMMA colloidal crystals were formed by centrifuging the PMMA colloid at 1500 rp, for 24 hours, decanting the water, and allowing the solid to dry for 3 days.

In the third step, various polymer precursors can be infiltrated into the silica- or latex-based colloidal crystals including monomers that were subsequently polymerized thermally or under UV. This approach can be used to create various porous polymer replicas, such as polystyrene (PS), poly(methyl (meth)acrylate) (PMMA), or polyurethane (see, Chem. Mater. 1998, 10, 1745; Chem. Mater. 1999, 11, 2827; and J. Am. Chem. Soc. 1999, 121, 11630, each of which is incorporated herein by reference), polymerized divinylbenzene (DVB) and ethylene glycol dimethylacrylate (EGDMA) (see, Science 1999, 283, 963; which is incorporated herein by reference), epoxy resins (see, Adv. Mater. 1998, 10, 1045; which is incorporated herein by reference), polydimethylsiloxane (PDMS) elastomers, (see, Adv. Mater. 2003, 15, 892; which is incorporated herein by reference), polyethylene using gaseous phase as the precursors by chemical vapor deposition (see Polymer 2008, 49, 5446; which is incorporated herein by reference), or poly(carbazole) via colloidal template-assisted electropolymerization (see, Adv. Mater. 2011, 23, 1287; which is incorporated herein by reference).

In the final step, the spherical polymer latex particles or silica spheres are removed by selective dissolution in organic solvents or hydrofluoric acid, respectively.

Monodisperse poly(methyl methacrylate) (PMMA) spheres were synthesized by surfactant-free emulsion polymerization of MMA in water with an azo initiator (2,2′-azobis(2-amidinopropane) dihydrochloride, V50) at 75° C. (See, Prog. Colloid Polym. Sci. 1976, 60, 163; which is incorporated herein by reference.) To create the colloidal crystal template, the colloidal suspension was subjected to centrifugation and drying, inducing the PMMA latex spheres to form three-dimensionally ordered arrays. The initial exemplary PMMA spheres had an average diameter of 480 nm with a narrow size distribution, and the spheres formed a close-packed into a face-centered cubic (f.c.c.) lattice. The colloidal crystals are predominantly f.c.c. with a small fraction of hexagonal close-packing (h.c.p.) or random close-packing (r.c.p.) regions. (See, Angew. Chem. Int. Ed. 2009, 48, 6212; which is incorporated herein by reference.) This phenomenon originates from the fact that f.c.c. is entropically favored over h.c.p. by ˜0.005RT per mol. (See, Nature 1997, 385, 141; which is incorporated herein by reference.) The f.c.c. component was induced by gravity and centrifuge-induced stresses, since only random stacking of hexagonally close-packed (r.h.c.p.) structure can form in microgravity. (See, Nature 1997, 387, 883; which is incorporated herein by reference.) The colloidal spheres in the colloidal crystals have a packing density of 0.74, which is the highest among the common crystal structures. (See, Soft Matter 2005, 1, 265; which is incorporated herein by reference.) In the third step of the preparation of the 3DOM polymeric materials, the PMMA colloidal crystals were used as the templates to create ordered macroporous polymeric materials, then, the void areas within the colloidal crystals were infiltrated with the desired mixture of monomers prior to in situ copolymerization.

The functional 3DOMs can also be employed for synthesis of nucleic acids, enzyme immobilization, as an attachment for a protein scaffold, or employed for purification proteins or analyte enrichment. For analyte capture specific antibodies or other proteins capable of molecular recognition are immobilized onto the 3DOM and the functionalized 3DOM can be added to aqueous or organic solutions to selectively capture the desired analyte from solution.

In another embodiment the three-dimensionally ordered macroporous hydrogel can be employed for degradation of biologically active agents in wastewater since the accessible functional groups on the hydrogel can be modified to contain functionalities that remove metal cations, radionuclides, dyes, anions and other miscellaneous pollutants from water, while the hydrogel can be readily packed into columns thereby increasing the rate of purification compared to membranes. The desired functionalities have been incorporated into membranes and are currently used/being evaluated on large scale. The functionalized hydrogels would be much easier to operate, and at a lower cost than membranes.

In a further embodiment of the invention the pores in the first formed colloidal crystal templated macroporous hydrogel can be preserved during drying and the resulting porous materials could be reformed by exposing the powder to a selected solvent, optionally comprising additional desired reagents, to swell and allow added agents to interact with functionality incorporated into the hydrogel during synthesis or in a post fabrication functionalization reaction.

### EXAMPLES

The examples that follow are intended to further describe certain non-limiting embodiments, without restricting the scope of the present invention. Persons having ordinary skill in the art will appreciate that variations of the following examples are possible within the scope of the invention, which is defined solely by the claims.

The following series of experiments are exemplary reactions and should not be considered to limit the scope of the reaction conditions or reagents that can be incorporated into the formed 3DOM's.

In order to synthesize colloidal crystals with diameters over 200 nm, spherical polystyrene particles were prepared, since literature reports PS are more controllable than PMMA over the particle size. The first step towards polystyrene colloidal crystals is depicted below.

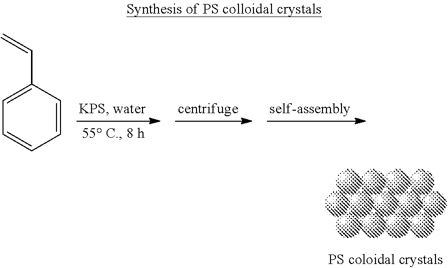

PS spheres were synthesized from mixtures with a composition of 250 mL of water, 15 mL of styrene, and 0.2 g of potassium persulfate (KPS, has a 10 hour half-life decomposition temperature of 55° C. in water) as the initiator. Water and styrene were added to a three-neck round-bottom flask, to which was attached a water-cooled condenser. The mixture was stirred at 350 rpm, while being heated to 55° C. and purged with nitrogen gas. After stabilization of the temperature at 55° C., the KPS in 3 mL of water was added, and the reaction was allowed to proceed for 8 h, producing PS spheres. The colloidal polymer was filtered through cottons to remove any large agglomerates then PS colloidal crystals were formed by centrifuging the colloid at 1500 rpm for 24 h, decanting the water, and allowing the solid to dry over 3 days.

### Example 1. Preparation of 3DOM Hydrogel by Colloidal Crystal Templating Via ATRP

Aqueous ATRP of oligo(ethylene glycol) methyl ether (meth)acrylate (OEOMA) was carried out in the presence of a PMMA colloidal crystal template following the conditions previously determined as suitable for the aqueous ATRP of OEOMA without templates (see, Macromolecules 2012, 45, 6371, which is incorporated by reference herein), as illustrated in Scheme 3.

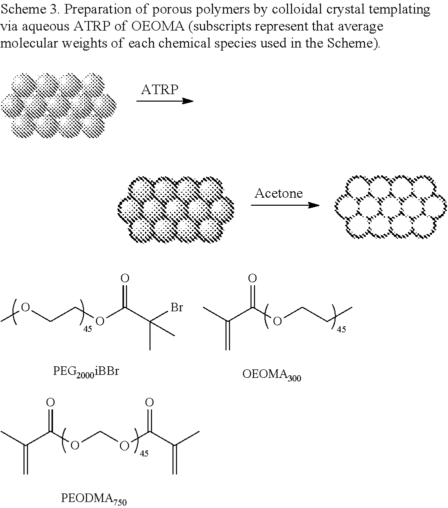

A series of aqueous ATRP reactions were carried out with systematically varied conditions to determine optimal conditions for ATRP of monomers and crosslinkers, e.g., poly(ethylene glycol) (meth)acrylate (PEOMA) and poly(ethylene oxide) di(meth)acrylate (PEODMA). (FIG. 1)

Three different ratios of reagents were evaluated;

(a) [PEG₂₀₀₀iBBr]/[OEOMA₃₀₀]/[PEODMA₇₅₀]/[CuCl]/[CuCl₂]/[bpy]=1/120/8/1/9/21, monomer/water=½ (w/w), 25° C., 5 h. (7%)

(b) [PEG₂₀₀₀iBBr]/[OEOMA₃₀₀]/[PEODMA₂₅₀]/[CuCl]/[CuCl₂]/[bpy]=1/120/25/1/9/21, monomer/water=½ (w/w), 25° C., 5 h. (21%)

(c) [PEG₂₀₀₀iBBr]/[OEOMA₃₀₀]/[PEODMA₂₅₀]/[CuCl]/[CuCl₂]/[bpy]=1/120/45/1/9/21, monomer/water=½ (w/w), 25° C., 5 h. (38%).

The subscripts represent that average molecular weights of each chemical species.

Conditions developed generally followed this procedure: 0.5 g of PMMA colloidal crystals was placed in a 20 mL glass vial sealed with rubber stopper. The vial was placed under vacuum for 5 min and then purged with nitrogen. The vacuum/purge cycle was repeated for five times. The aqueous solution of a mixture of catalyst (CuCl/CuCl₂/bipyridine), monomer and crosslinker was added via gastight syringe to the vial. The polymerization was carried out at 25° C. for 5 h. The PMMA template was removed from the sample by extraction with acetone over a period of one day.

### Example 2. Preparation of 3DOM Hydrogel by Colloidal Crystal Templating Via Conventional Free Radical Polymerization (FRP)

A series of FRP reactions were carried out with systematically varied conditions to determine optimal conditions for FRP of monomers and crosslinkers, e.g., poly(ethylene glycol) (meth)acrylate (PEOMA) and poly(ethylene oxide) di(meth)acrylate (PEODMA). Conditions developed generally followed this procedure: 0.5 g of PMMA colloidal crystals was placed in a 20 mL glass vial sealed with rubber stopper. The vial was put in vacuum for 5 min and then purged with nitrogen. The vacuum/purge was repeated for five times. The monomer and crosslinker mixture aqueous solution was added via gastight syringe to the vial. The polymerization was carried out at 80° C. for 18 h. The PMMA template was removed from the sample by extraction with acetone for one day.

As noted in the background to the invention there are several hydrophilic monomers that can be incorporated into such 3DOM hydrogels by colloidal crystal templating copolymerization of free radically copolymerizable monomers utilizing reversible deactivation radical polymerizations (RDRP) procedures, herein exemplified by ATRP, or by other FRP procedures. Of particular utility would be acrylamide monomers, such as N,N-methylenebisacrylamide which are stable under basic conditions.

### Example 3. Preparation of Trypsin Grafted Hydrogel. (Scheme 1)

An exemplary 3DOM hydrogel prepared by copolymerization of PEOMA and PEODMA was mixed with succinic anhydride, triethylamine, and N,N-(dimethylamino)pyridine (DMAP) in anhydrous acetone. Succinic anhydride (1.40 g, 14 mmol), triethylamine (0.14 g, 1.4 mmol), and N,N-(dimethylamino)pyridine (DMAP, 17 mg, 0.14 mmol) were dissolved in 4 mL of anhydrous acetone. PEODMA-PEO-OH-526 (1.0 g, ca. 1.4 mmol —OH group) was taken out of acetone and added into the solution. The mixture was shaken for 1 d at 60° C. and then was washed with excess acetone. TRYPSIN (0.3 g, minimum 2500 USP units/mg), N-(3-Dimethylaminopropyl)-N′-ethylcarbodiimide hydrochloride (EDC.HCl, 0.28 g, 1.5 mmol), and N-hydroxysuccinimide (0.17 g, 0.15 mmol) were dissolved into 4 mL TRIS (PH=8.0) solution. PEODMA-PEO-COOH-526 (1.0 g, ca. 1.5 mmol —COOH group) was taken out of acetone and added into the solution. The mixture was shaken for 1 d at room temperature. The product was washed with TRIS (PH=8.0).

### Example 3B) Calculation of % Trypsin in Hydrogel

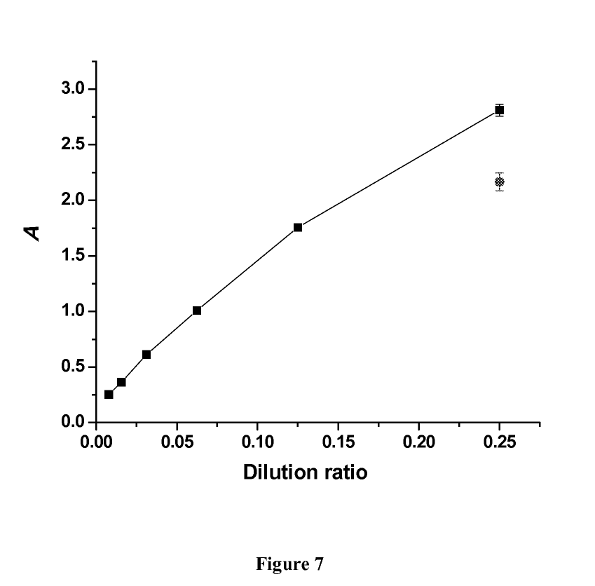

A BCA protein assay kit was used to generate a calibration curve for the determination of residual trypsin concentration in the reaction mixture. Pierce BCA protein assay reagent (bicinchoninic acid) was purchased from Thermo Fisher Scientific Inc. A trypsin stock solution (19.0 mg/mL) was prepared by dissolving 38.0 mg of trypsin, 40.8 mg of EDC.HCl, and 24.5 mg of NHS in 2.0 mL of TRIS buffer solution. The trypsin stock solution was diluted to 0.25, 0.125, 0.0625, 0.03125, 0.01563, and 0.00781 of its original concentration by adding TRIS buffer solution. 25 μL of every diluted sample were transferred into a 96 flat bottom transparent polystyrol well plate. BCA protein assay kit solutions B and A were mixed in a ratio of 1/50 (v/v). 200 μL of the resulting BCA assay mixture was added to each sample in the wells. The samples were incubated at 37° C. for 30 min, and the absorption at 562 nm was measured using a TECAN infinite M1000 plate reader. The obtained calibration curve was shown in FIG. 7. Then, 100 μL of the final reaction mixture was taken and diluted to 0.25 of its original concentration by adding TRIS buffer solution. 25 μL of the diluted sample was transferred into a 96 flat bottom transparent polystyrol well plate. BCA protein assay kit solutions B and A were mixed in a ratio of 1/50 (v/v). 200 μL of the resulting BCA assay mixture was added to the sample in the well. The samples were incubated at 37° C. for 30 min, and the absorption at 562 nm was measured using a TECAN infinite M1000 plate reader. The concentration of trypsin in the final reaction mixture was 14.63 mg/mL according to the calibration curve was shown in FIG. 7. The concentration of trypsin in the original reaction mixture (2.0 mL) was 19.0 mg/mL, so the amount of trypsin on the hydrogel was 8.74 mg for 38 mg of hydrogel, or 18.7 wt. %.

### Example 3C: Leaching Experiment

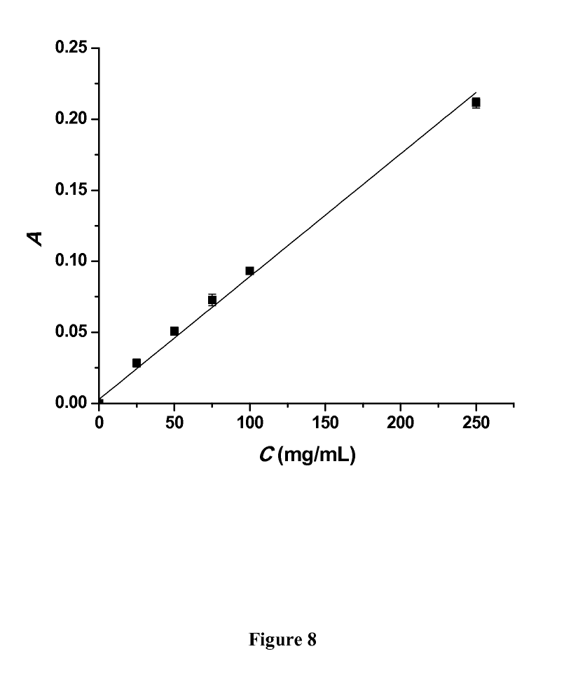

For the determination of trypsin concentration in the solution, a BCA protein assay kit was used to generate a calibration curve. A trypsin stock solution (2.0 mg/mL) was prepared by dissolving 20.0 mg of trypsin in 10.0 mL of TRIS buffer solution. The calibration curve was measured using the trypsin concentrations outlined in Table 1. 25 μL of every sample were transferred into a 96 flat bottom transparent polystyrol well plate. BCA protein assay kit solutions B and A were mixed in a ratio of 1/50 (v/v). 200 μL of the resulting BCA assay mixture was added to each sample in the wells. The samples were incubated at 37° C. for 30 min, and the absorption at 562 nm was measured using a TECAN infinite M1000 plate reader. The obtained calibration curve was shown in FIG. 8.

| | | | | | | |
|---|---|---|---|---|---|---|
| Trypsin concentration (μg/mL) | 0 | 25 | 50 | 75 | 100 | 250 |
| TRIS buffer solution (μL) | 1000 | 987.5 | 975 | 962.5 | 950 | 875 |
| Trypsin stock solution (μL) | 0 | 12.5 | 25 | 37.5 | 50 | 125 |

The sample hydrogel-trypsin was suspended in TRIS buffer solution for 1 d. A previously acquired calibration curve (FIG. 8) was used to quantify the amount of enzyme leached out the sample. The supernatant was measured by BCA protein assay and no absorption at 562 nm was observed, indicating that trypsin leaching had not occurred.

### Example 4. Determination of the Activity of the Trypsin Grafted Hydrogel

A 2 mM N-benzoyl-L-argininep-nitroanilide (BAPNA) solution was prepared by dissolving 4.4 mg of BAPNA in 0.1 mL of DMSO and diluted to 25 mL tris buffer (50 mM, pH 8). 0.2 mL of the BAPNA solution (2 mM) was mixed with 2 mL of tris buffer (50 mM, pH 8) in a UV quartz cuvette, hydrogel-trypsin (0.5 mg) was added into the mixture and UV spectra were measured periodically. The results are shown in FIG. 4 where the fact that the hydrolysis was successful is demonstrated by the increased intensity of the cumulative UV-vis spectra recorded over a period of 3 hours

### Example 5. Hydrogel with Three-Dimensionally Ordered Macroporous Structure for Protein Digestion

A novel three-dimensionally ordered macroporous (3DOM) hydrogel with immobilized-enzyme was synthesized, characterized, and used for protein digestion. The 3DOM hydrogel was prepared by the copolymerization of poly(ethylene glycol) (meth)acrylate (PEOMA) and poly(ethylene oxide) di(meth)acrylate (PEODMA) in the presence of latex colloidal crystal as the template. The colloidal crystal was synthesized by surfactant-free emulsion polymerization, and has a uniform pore size typically in the range of 100-1000 nm. After being used as the sacrificing template, the colloidal crystal was dissolved in acetone to generate the 3D ordered macropores with interconnected windows in the crosslinked hydrogel, which facilitated the liquid transport through the pores. The trypsin was introduced onto the pore surfaces through condensation reactions. The structure of the functionalized 3DOM hydrogel was characterized by scanning electron microscopy (SEM) and nanoscale 3D X-ray microscopy (XRM). It was demonstrated that the porous structure of the hydrogel was able to undergo reversible shrinkage and expansion by drying and swelling. The trypsin-immobilized hydrogel was loaded in a column and showed high activity for enzyme digestion when an aqueous solution of Nα-benzoyl-L-arginine p-nitroanilide (BAPNA) or bovine serum albumin (BSA) passing through it. This study indicates that the colloidal crystal templated 3DOM hydrogel is a useful enzyme immobilization substrate for protein digestion. (See Dimensionally Ordered Macroporous Structure for Protein Digestion, Hongkun He, Saadyah Averick, Pratiti Mandal, Shawn Litster, Jeff Gelb, Naomi Kotwal, Arno Merkle, and Krzysztof Matyjaszewski, 2013 Materials Research Society Fall Meeting & Exhibit, Symposium E, Dec. 2, 2013, Boston, Mass.; downloaded from http://www.mrs.org/fall-2013-program-e/; downloaded on Nov. 18, 2014, which is hereby incorporated by reference herein.)

What is claimed is:

1. A three-dimensionally ordered macroporous hydrogel, comprising:
a polymer comprising at least one hydrophilic monomer, and at least one crosslinker;
wherein the polymer comprises a system of interconnected pores, the interconnected pores comprising a uniform pore size in the range of 50 to 5000 nm; and
a plurality of first pore functional groups;
wherein the plurality of first pore functional groups is selected to covalently bond with a selected bioresponsive molecule.

2. The three-dimensionally ordered macroporous hydrogel of claim 1, wherein the at least one hydrophilic monomer is selected from the group consisting of (ethylene glycol) (meth)acrylate, hydroxylated-(ethylene glycol) (meth)acrylate, quaternized 2-(dimethylamino)ethyl (meth)acrylate, hydroxyalkyl (meth)acrylates, n-vinyl pyrrolidone, and acrylamides.

3. The three-dimensionally ordered macroporous hydrogel of claim 1, wherein the at least one crosslinker comprises a monomeric unit that is selected from the group consisting of (ethylene glycol) di(meth)acrylate, hydroxylated-(ethylene glycol) di(meth)acrylate, quaternized 2-(dimethylamino)ethyl di(meth)acrylate, a hydroxyalkyl di(meth)acrylate, and a diacrylamide.

4. The three-dimensionally ordered macroporous hydrogel of claim 1, wherein prior to crosslinking, the at least one crosslinker comprises two or more vinyl groups.

5. The three-dimensionally ordered macroporous hydrogel of claim 4, wherein the at least one crosslinker is selected from the group consisting of diethylene glycol di(meth)acrylate, poly(ethyleneoxide) di(meth)acrylate, trimethylolpropane tri(meth)acrylate, a propylene glycol di(meth)acrylate, a diacrylate of hydrophilic polymer, a diacrylate of caprolactone modified hydroxy pivalic acid neopentyl glycol ester, a polyethoxified tetramethylol methane tetraacrylate, a diacrylate, neopentyl glycol di(meth)acrylate, stearyl diacrylate, 1,4-butane diol di(meth)acrylate, and bis(2-methacyloyloxyethyl) disulfide.

6. The three-dimensionally ordered macroporous hydrogel of claim 4, wherein the crosslink density in the matrix of the hydrogel comprises between 1-100% of the vinyl units present in the hydrogel.

7. The three-dimensionally ordered macroporous hydrogel of claim 1, wherein the uniform pore size is in the range of 100 to 1000 nm.

8. The three-dimensionally ordered macroporous hydrogel of claim 1, wherein the plurality of first pore functional groups is selected from the group consisting of a hydroxyl group, a carboxyl group, an amino group, a mercapto group, a nitro group, a cyano group, an azido group, an alkyl group, a halogenoalkyl group, an alkenyl group, an alkenyloxy group, an alkynyl group, an alkoxy group, an alkylthio group, a formyl group, an alkanoyl group, an alkyloxycarbonyl group, an oxo group, an urea group, a thiourea group, an aminoalkyl group, an aryl group, an aralkyl group, an aryloxy group, an arylthio group, an alkylsulfonyl group, an arylsulfonyl group, a carbamoyl, a heterocyclic group, a protected amino, a protected hydroxyl, and a protected carboxyl group.

9. The three-dimensionally ordered macroporous hydrogel of claim 1, where at least one of the plurality of first pore functional groups can be utilized to form a covalent bond with a selected bioresponsive molecule.

10. The three-dimensionally ordered macroporous hydrogel of claim 1, where the plurality of first pore functional groups can be converted to a plurality of second pore functional groups that are utilized to form a covalent bond with a selected bioresponsive molecule.

11. The three-dimensionally ordered macroporous hydrogel of claim 1, where the plurality of first pore functional groups can be converted to a plurality of one or more differing second pore functional groups that are utilized to form covalent bonds with one or more selected bioresponsive molecules.

12. The three-dimensionally ordered macroporous hydrogel of claim 1, where a fraction of first pore functional groups can be converted to a plurality of one or more differing second pore functional groups and the formed mixture of functional groups are utilized to form covalent bonds with one or more selected bioresponsive molecules.

13. The three-dimensionally ordered macroporous hydrogel of claim 10, wherein at least one of the first pore functional groups or at least one of the second pore functional groups is covalently bonded with a bioresponsive molecule comprising an enzyme.

14. The three-dimensionally ordered macroporous hydrogel of claim 13, wherein the bioresponsive molecule comprises trypsin, papain protein G or synthetically relevant agents exemplified by Lipase.

15. The three-dimensionally ordered macroporous hydrogel of claim 13, wherein the bioresponsive molecule comprises trypsin.

16. The three-dimensionally ordered macroporous hydrogel of claim 10, wherein the plurality of first or second pore functional groups is covalently bonded with a bioresponsive molecule to form a protein scaffold.

17. The three-dimensionally ordered macroporous hydrogel of claim 10, wherein the plurality of first or second pore functional groups is covalently bonded with a bioresponsive molecule for protein purification.

18. The three-dimensionally ordered macroporous hydrogel of claim 10, wherein the plurality of first or second pore functional groups is covalently bonded with a bioresponsive molecule for solid phase synthesis.

19. The three-dimensionally ordered macroporous hydrogel of claim 10, wherein the plurality of first or second pore functional groups is covalently bonded with a bioresponsive molecule for nucleic acid synthesis.

20. The three-dimensionally ordered macroporous hydrogel of claim 10, wherein the plurality of first or second pore functional groups is covalently bonded with a bioresponsive molecule for polypeptide synthesis.

21. The three-dimensionally ordered macroporous hydrogel of claim 10, wherein the plurality of first or second pore functional groups is covalently bonded with a bioresponsive molecule for analyte detection.

22. The three-dimensionally ordered macroporous hydrogel of claim 10, wherein the plurality of first or second pore functional groups is covalently bonded with a bioresponsive molecule for adsorption of analytes and measuring analyte concentrations.

23. The three-dimensionally ordered macroporous hydrogel of claim 10, wherein the plurality of first or second pore functional groups is covalently bonded with a bioresponsive molecule for organic synthesis.

24. The three-dimensionally ordered macroporous hydrogel of claim 10, wherein the plurality of first or second pore functional groups is covalently bonded with a bioresponsive molecule for degradation of biologically active agents in wastewater.

25. (canceled)

25.-45. (canceled)

46. A method of preparing a three-dimensionally ordered macroporous hydrogel, comprising:
preparing a colloidal crystal template, comprising:
providing a plurality of spherical particles, the particles having a uniform particle size distribution and having an average particle size diameter in the range of 10 nm to 100 μm;
assembling the spherical particles into a colloidal crystal template;
wherein the assembling comprises a process of one or more of sedimentation, centrifugation, electro deposition, vertical deposition, filtration, and slit filling; and
wherein the colloidal crystal template comprises an ordered and repeating array of the spherical particles defining a uniform array of pores between contacting spherical particles, having an average pore size in the range of 50 to 5000 nm;
infiltrating polymer precursors into the pores of the colloidal crystal template;
wherein the polymer precursors comprise at least one hydrophilic monomer and at least one crosslinker;
wherein at least one of the polymer precursors comprises a first pore functional group that can form covalent bonds with a selected bioresponsive molecule;
polymerizing the polymer precursors within the pores of the colloidal crystal template; and
selectively removing the colloidal crystal template.

47. The method of claim 46, wherein the plurality of spherical particles comprises silica particles.

48. The method of claim 46, wherein the plurality of spherical particles comprises polymeric particles.

49. The method of claim 48, wherein the plurality of spherical particles comprises particles prepared by a surfactant free emulsion polymerization.

50. The method of claim 48, wherein the plurality of spherical particles comprises one of polystyrene (PS) particles and poly(methyl (meth)acrylate) (PMMA) particles.

51. The method of claim 48, wherein the plurality of spherical particles comprises poly(methyl (meth)acrylate) (PMMA) particles.

52. The method of claim 46, wherein the assembling the spherical particles into a colloidal crystal template step comprises centrifuging the spherical particles.

53. The method of claim 46, wherein the at least one hydrophilic monomer is selected from the group consisting of (ethylene glycol) (meth)acrylate, hydroxylated-(ethylene glycol) (meth)acrylate, quaternized 2-(dimethylamino)ethyl (meth)acrylate, hydroxyalkyl (meth)acrylates, n-vinyl pyrrolidone, and acrylamides.

54. The method of claim 46, wherein the at least one crosslinker is selected from the group consisting of (ethylene glycol) di(meth)acrylate, hydroxylated-(ethylene glycol) di(meth)acrylate, quaternized 2-(dimethylamino)ethyl di(meth)acrylate, a hydroxyalkyl di(meth)acrylate, and a diacrylamide.

55. The method of claim 46, wherein the at least one crosslinker is selected from the group consisting of diethylene glycol di(meth)acrylate, poly(ethyleneoxide) di(meth)acrylate, trimethylolpropane tri(meth)acrylate, divinylbenzene, a propylene glycol di(meth)acrylate, a diacrylate of hydrophilic polymer, a diacrylate of caprolactone modified hydroxy pivalic acid neopentyl glycol ester, a polyethoxified tetramethylol methane tetraacrylate, a diacrylate, neopentyl glycol di(meth)acrylate, stearyl diacrylate, 1,4-butane diol di(meth)acrylate, and bis(2-methacyloyloxyethyl) disulfide.

56. The method of claim 46, wherein the step of infiltrating polymer precursors into the colloidal crystal template comprises infiltrating the colloidal crystal template with polymeric precursors required for a controlled radical polymerization (CRP).

57. The method of claim 46, wherein the step of infiltrating polymer precursors into the colloidal crystal template comprises infiltrating the colloidal crystal template with polymeric precursors required for an atom transfer radical polymerization reaction, the polymeric precursors comprising at least one hydrophilic monomer, at least one crosslinker, an initiator, a transition metal catalyst having two accessible oxidation states that are separated by one electron, and a ligand capable of forming a ligand-transition metal catalyst complex; and wherein the polymerizing step comprises an atom transfer radical polymerization (ATRP).

58. The method of claim 57, wherein the step of infiltrating polymer precursors into the colloidal crystal template comprises infiltrating the colloidal crystal template with an aqueous solution comprising a brominated poly(ethylene glycol) initiator (PEG), oligo(ethylene glycol) methyl ether (meth)acrylate (OEOMA) monomer, poly(ethylene oxide) di(meth)acrylate (PEOMA) crosslinker, cuprous halide (CuX), cupric chloride (CuX₂), and a ligand (L) forming a soluble complex with the transition metal catalyst.

59. The method of claim 58, wherein the molar ratios of PEG/OEOMA/PEOMA/CuX/CuX₂/L range from 1/120/8/1/9/21 to 1/120/45/1/9/21, and wherein the monomer to initiator ratio is in a range of 10-10,000 to 1.

60. The method of claim 46, wherein the step of infiltrating polymer precursors into the colloidal crystal template comprises infiltrating the colloidal crystal template with polymeric precursors required for a free radical polymerization reaction.

61. The method of claim 60, wherein the at least one monomer comprises poly(ethylene glycol (meth)acrylate (PEOMA) and the at least one crosslinker comprises poly(ethylene oxide) di(meth)acrylate (PEODMA).

62. The method of claim 46, further comprising a comonomer selected from the group consisting of a substituted styrene, a (meth)acrylate, an acrylamide, and a vinyl pyrrolidone.

63. The method of any of claim 46, further comprising covalently bonding a plurality of bioresponsive molecules to the first pore functional group of the three-dimensionally ordered macroporous hydrogel.

64. The method of claim 63, wherein the bioresponsive molecule comprises trypsin.

65. The method of claim 46, wherein selectively removing the colloidal crystal template comprises dissolving the colloidal crystal template in a solvent, wherein the solvent does not solubilize the three-dimensionally ordered macroporous hydrogel.

66. The method of claim 65, wherein the solvent comprises hydrofluoric acid.

67. The method of claim 65, wherein the solvent comprises one or more of acetone, tetrahydrofuran, and a solution of acetone and tetrahydrofuran.
